(19) United States
(12) Patent Application Publication
(10) Pub. No.: US 2007/0232989 A1
(43) Pub. Date: Oct. 4, 2007

(54) Liquid Co-Infusion Device

(75) Inventors: Ichiro Kitani, Fukuroi-shi (JP); Shigeaki Funamura, Fukuroi-shi (JP)

Correspondence Address: TYCO HEALTHCARE - EDWARD S. JARMOLOWICZ, 15 HAMPSHIRE STREET, MANSFIELD, MA, 02048, US

(73) Assignee: NIPPON SHERWOOD MEDICAL INDUSTRIES LTD., Tokyo (JP)

(21) Appl. No.: 11/680,349

(22) Filed: Feb. 28, 2007

(30) Foreign Application Priority Data
Mar. 3, 2006 (JP) 2006-058262

(51) Int. Cl. A61M 1/00

(52) U.S. Cl. 604/28

(57) ABSTRACT

A liquid co-infusion device has a plurality of connection ports. The device includes an operating part axially movable within a chamber of the device so as to selectively isolate one of said connection ports from the chamber. An end of the operating part may include a groove or through channel movable to cause such isolation. Preferably, the axial movement of the operating part is guided so as to include a axial movement stop.

### FIELD OF THE INVENTION

The present invention generally relates to a liquid co-infusion device.

### BACKGROUND OF THE INVENTION

In the past, a specific physiological saline, liquid medicine or the like would have been administered to a patient using multiple transfusion tubes. In such cases, a liquid co-infusion device would be used and the operation carried out by connecting or disconnecting the various transfusion tubes. Among such liquid co-infusion devices are those that have three branch tubes. The liquid co-infusion device is constituted with a chamber part that connects to the three branch tubes and a valve body that is rotated inside the chamber part by operating an operating part. By operating the operating part to rotate the valve body, any of the branch tubes can be connected or disconnected.

However, with the abovementioned liquid co-infusion device, the operating part provided projects from the circumferential surface of the chamber part, and the tube members connected to the branch tubes sometimes get tangled on the operating part, or the patient sometimes touches the operating part and turns it. For this reason, a liquid co-infusion device that can switch among branch tube channels has been developed such that the chamber part is cylindrical, with the operating part not projecting from the circumferential surface of the chamber part, by the operating part being movable in the axial direction of the chamber part to connect or disconnect a specific branch tube to or from the chamber part (for example, see Japanese Kokai Patent Application No. Sho 62[1987] 172962). This liquid co-infusion device is constituted such that two branch tubes are furnished, one on each side of the circumferential surface of a cylindrical chamber part, so that the two branch tubes can be opened and closed by rotating the valve body around its axis inside the chamber part.

However, this liquid co-infusion device has only two branch tubes and can only connect or disconnect two branch tubes. Therefore, with this liquid co-infusion device, it is not possible to connect multiple transfusion tubes or the like used for medical treatment, and to switch from connecting or disconnecting the various transfusion tubes. There is also the problem that the valve body must be rotated to connect or disconnect the two branch tubes, so that operating it is difficult.

### SUMMARY OF THE INVENTION

In one aspect of the present invention, a liquid co-infusion device having a body generally comprises a chamber having a first connection port formed at one end in an axial direction of the chamber and second and third connection ports extending sidewards from the chamber. An operating part is arranged at a second end in the axial direction of the chamber and is movable in the axial direction of the chamber. Movement of the operating part in the axial direction switches between connection and disconnection of one of the connection ports to an interior of the chamber.

Other features will be in part apparent and in part pointed out hereinafter.

### BRIEF DESCRIPTION OF THE DRAWINGS

Embodiments of the invention will now be described by way of example only with reference to the accompanying figures in which:

FIG. 1(a) is a plan view of an operating part of a liquid co-infusion device pertaining to a first embodiment of the present invention when in the up position;

FIG. 1(b) is a front view of the operating part of FIG. 1(a) in the up position;

FIG. 1(c) is a side view of the operating part of FIG. 1(a) in the up position;

FIG. 2(a) is a plan view of the operating part of the liquid co-infusion device when in the down position;

FIG. 2(b) a front view of the operating part of FIG. 2(a) in the down position;

FIG. 2(c) a side view of the operating part of FIG. 2(a) in the down position;

FIG. 3 is a cross section at 3-3 in FIG. 1(a);

FIG. 4 is a cross section at 4-4 in FIG. 1(a);

FIG. 5 is a cross section at 5-5 in FIG. 2(a);

FIG. 6 is a cross section at 6-6 in FIG. 2(a);

FIG. 7 is a cross section at 7-7 in FIG. 2(b);

FIG. 8 is a cross section showing a liquid co-infusion device to which an adaptor is attached and in which the operating part is in the up position, viewed from the front;

FIG. 9 is a cross side sectional view section showing a liquid co-infusion device to which an adaptor is attached and in which the operating part is in the up position, viewed from the side;

FIG. 10 is a cross front sectional view showing a liquid co-infusion device to which an adaptor is attached and in which the operating part is in the down position, viewed from the front;

FIG. 11 is a cross sectionside sectional view showing a liquid co-infusion device to which an adaptor is attached and in which the operating part is in the down position, viewed from the side;

FIG. 12(a) is a plan view of an shows the operating part of a liquid co-infusion device pertaining to a second embodiment of the present invention, when in the up position;

FIG. 12(b) is (a) is a plan view, (b) a front view of the operating part of FIG. 12(a) in the up position;

FIG. 12(c) is a side view of the operating part of FIG. 12(a) in the up position;

FIG. 13 is a front view showing the operating part of the liquid co-infusion device shown in FIG. 12, in the down position;

FIG. 14 is a cross section at 14-14 in FIG. 12(b);

FIG. 15 is a cross section at 15-15 in FIG. 12(a);

FIG. 16 is a cross section at 16-16 in FIG. 12(b);

FIG. 17 is a cross section at 17-17 in FIG. 13; and

FIG. 18 is a cross section showing the liquid co-infusion device in FIG. 13 viewed from the front.

Corresponding reference characters indicate corresponding parts throughout the drawings.

### DETAILED DESCRIPTION OF THE DRAWINGS

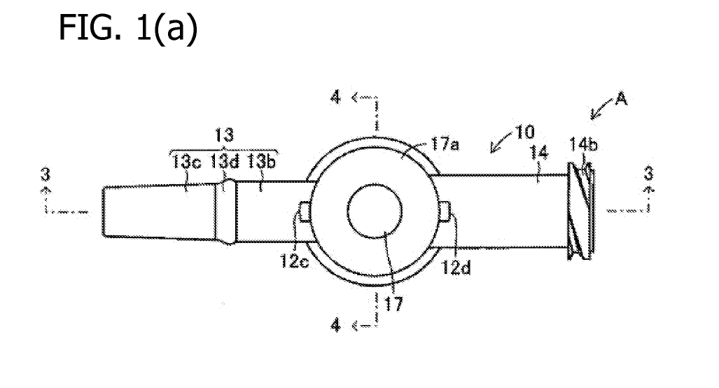

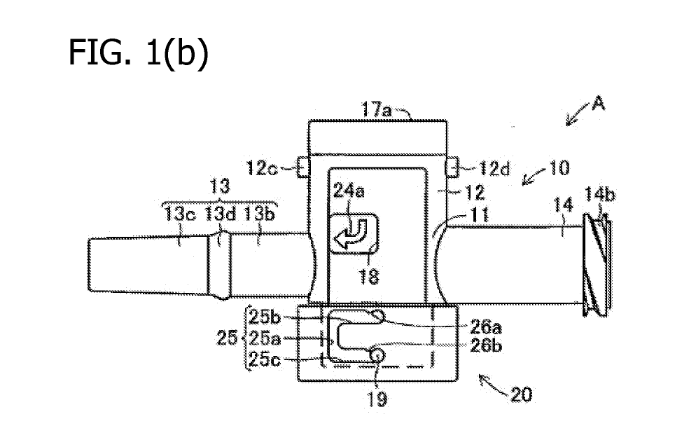

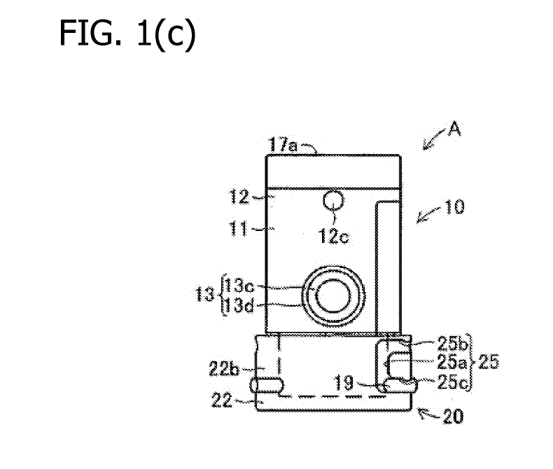

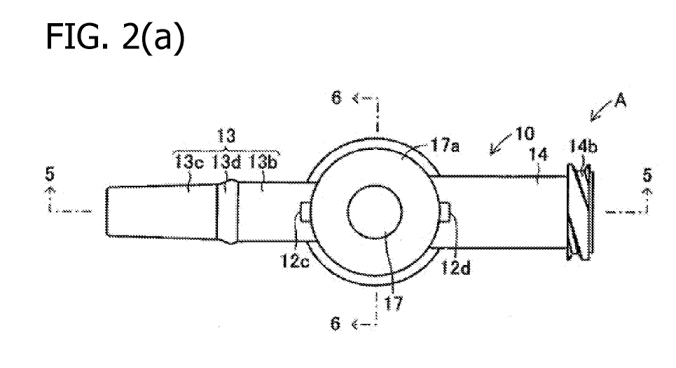

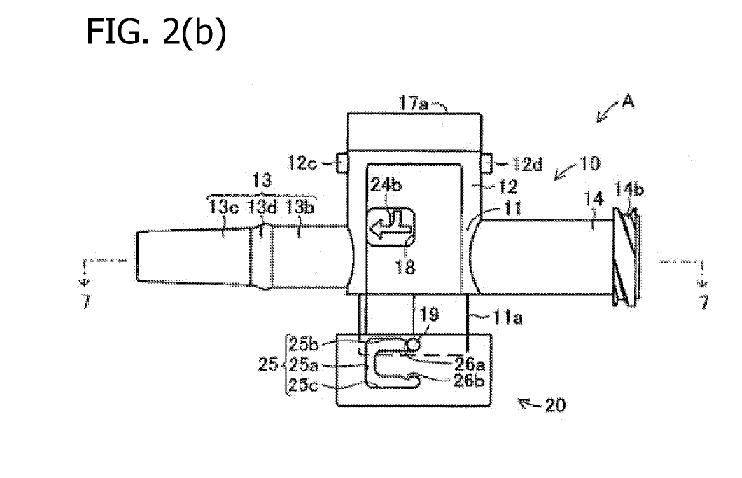

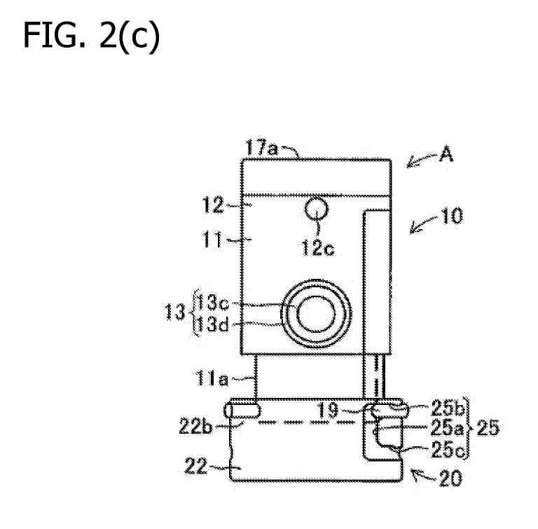

A liquid co-infusion device pertaining to one embodiment of the present invention will be explained in detail. FIGS. 1 and 2 show a liquid co-infusion device (A) pertaining to a first embodiment of the present invention. The liquid co-infusion device (A) is constituted with a liquid co-infusion device body (10) and an operating part (20). Liquid co-infusion device body (10) is constituted with a cylindrical chamber part (11) that is short in the axial direction, a flow merging branch tube (12) formed at one end (the top in FIGS. 1 and 2) in the axial direction of chamber part (11), and a downstream branch tube (13) and an upstream branch tube (14) that are connected on either side of the outer circumferential surface of chamber part (11) to extend along the same axis to maintain an angle of 180 degrees.

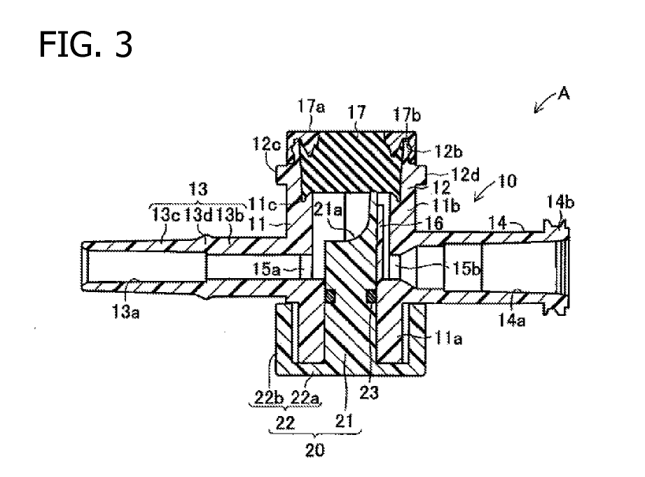

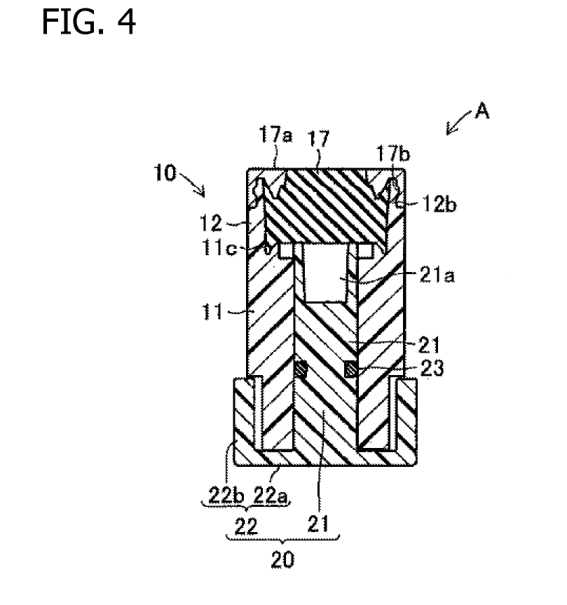

Chamber part (11) is formed as a stepped cylinder, such that the bottom section is narrower than the top part in the cross section viewed from the front surface as shown in FIG. 3, and the thickness is uniform in the cross section viewed from the side surface as shown in FIG. 4. Also, two through holes (15a) and (15b) are formed in the section facing the interface between small diameter part (11a) on the bottom and large diameter part (11b) on the top, this being positioned in the axial center of chamber part (11). Downstream branch tube (13) is furnished in the section of chamber part (11) corresponding to through hole (15a), and the inside of chamber part (11) and a channel (13a) formed inside downstream branch tube (13) are connected via through hole (15a).

Upstream branch tube (14) is formed in the section of chamber part (11) corresponding to through hole (15b), and the inside of chamber part (11) and channel (14a) formed inside upstream branch tube (14) are connected via through hole (15b). A vertical wall (16) to prevent backflow is also formed in the section of small diameter part (11a) of chamber part (11) corresponding to through hole (15b). Vertical wall (16) extends upward inside chamber part (11) from the top edge of the inner circumferential surface of small diameter part (11a), maintaining a separation from through hole (15b), so that medicine or the like flows through the top inside of chamber part (11) when medicine or the like flows from upstream branch tube (14) toward the inside of chamber part (11). Because of this, air or the like can be prevented from remaining in chamber part (11).

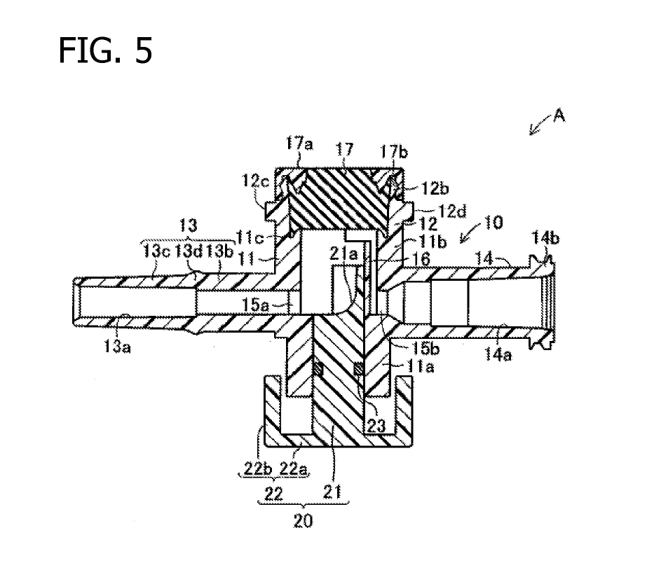

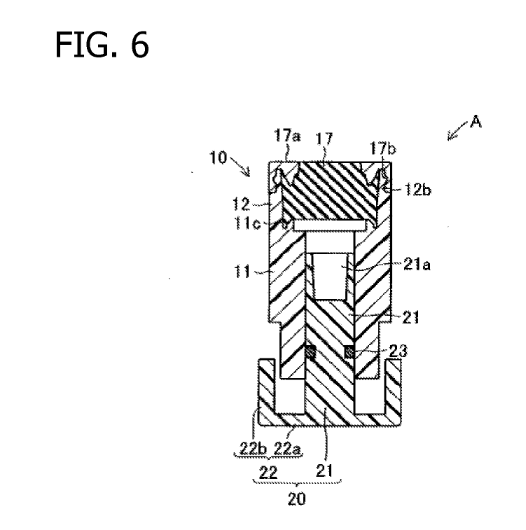

Therefore, with regard to vertical wall (16), when operating part (20) is as shown in FIGS. 5 and 6 (FIGS. 5 and 6 show valve body (21), described below, connecting chamber part (11) to upstream branch tube (14) by virtue of operating part (20) being in descended position), when medicine or the like is sent from upstream branch tube (14) toward the inside of chamber part (11), liquid rises along vertical wall (16) after passing through the inside of upstream branch tube (14) and through hole (15b) and spills over vertical wall (16) to flow into chamber part (11). In addition, the liquid flows to downstream branch tube (13) via through hole (15a).

Flow merging branch tube (12) formed at the top of chamber part (11) is formed in a cylindrical shape, the length of which in the axial direction is shorter than, and the diameter of which is larger than, downstream branch tube (13) or upstream branch tube (14), and it is constituted integrally with chamber part (11). A taper is furnished on the inner circumferential surface of flow merging branch tube (12) such that the diameter of the top opening is larger than the diameter toward chamber part (11). The internal diameter of flow merging branch tube (12) is larger than the internal diameter of chamber part (11), and a level difference is formed on the inside at the interface between the top end of chamber part (11) and the bottom end of flow merging branch tube (12). A shallow locking slot (11c) is formed along the outer circumference of the level difference, and a rubber stopper (17) made of natural rubber or synthetic rubber is inserted inside flow merging branch tube (12) into locking slot (11c), thus closing off the inside of flow merging branch tube (12).

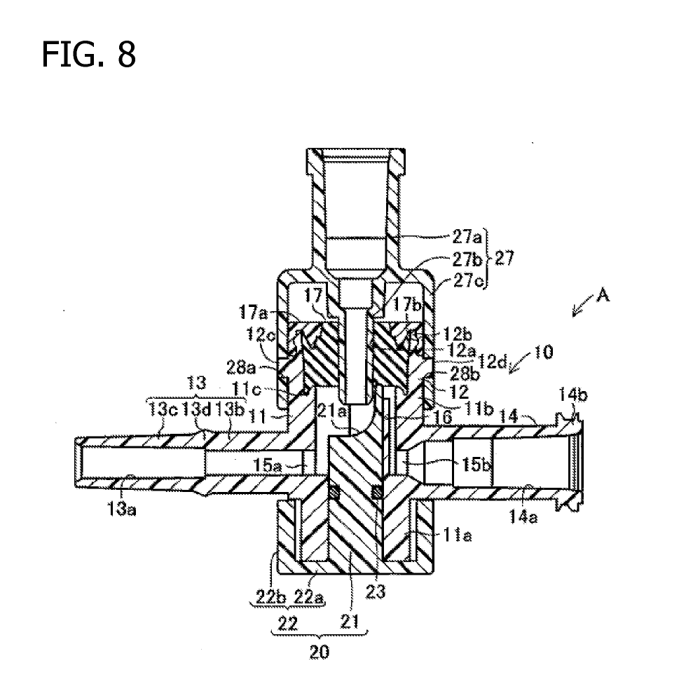

A slit (12a) (shown in FIGS. 8-11) that passes between the inside of chamber part (11) and the outside of flow merging branch tube (12) to form a channel in flow merging branch tube (12) is formed in rubber stopper (17). Slit (12a) is closed by the elasticity of rubber stopper (17) when flow merging branch tube (12) is not in use. A projecting part (12b), the outer circumferential surface of which is formed to be uneven, is also furnished on the top end of flow merging branch tube (12), and a cover (17a) that holds rubber stopper (17) in place in flow merging branch tube (12) is attached via projecting part (12b) in the spring part of flow merging branch tube (12).

Cover (17a) is formed approximately in a ring shape, the center of the top surface of which is open, and a locking recess (17b) that can lock onto projecting part (12b) is formed in the inner circumferential surface at the side. Therefore, cover (1 7a) presses the part of the top surface of rubber stopper (17) toward the outer circumference, and is attached in the opening of flow merging branch tube (12) by virtue of locking recess (17b) being locked onto projecting part (12b). A pair of locking projections (12c) and (12d) is also formed on the left and right side of the outer circumferential surface of flow merging branch tube (12) below projecting part (12b), and a small square window (18) is formed in the section of the circumferential surface of chamber part (11) somewhat toward downstream tube (13). In addition, a guiding projection (19) as the guiding part of the present invention is furnished below window (18) near the bottom end of the circumferential surface of chamber part (11).

Downstream branch tube (13) is formed integrally with chamber part (11) and is constituted with a base end (13b) positioned toward chamber part (11) and a male luer part (13c) positioned at the tip and formed to be narrower than base end (13b). Male luer part (1 3c) is formed with a pointed shape such that the tip is narrower than base end (13b). A projecting part (13d) is formed around on the outer circumferential surface of downstream tube (13) at the interface between base end (13b) and male luer part (13c).

Upstream branch tube (14) is formed integrally with chamber part (11), and a channel (14a) consisting of a tapered hole is formed inside. The channel (14a) is connected to through hole (15b), and the section toward through hole (15b) is tapered such that the diameter nearer through hole (15b) is smaller and the diameter farther from through hole (15b) is larger. The upstream section of channel (14a) is tapered such that the diameter gradually becomes larger closer to the opening in upstream branch tube (14). A threaded connecting part (14b) is formed on the outer circumference of the opening in upstream branch tube (14).

Operating part (20) is constituted with a valve body (21) that is approximately a round column, and a grip part (22) connected to the bottom end of valve body (21). Valve body (21) is disposed inside chamber part (11) and can move in the axial direction inside chamber part (11) by the operation of operating part (22). A slot for a seal member (23) is formed around the outer circumferential surface of valve body (21) in the section somewhat toward the bottom from the center. When valve body (21) moves inside chamber part (11), valve body (21) slides against the inner circumferential surface of small diameter part (11a) and vertical wall (16) and maintains a prescribed distance from the opening in channel (13a) of downstream branch tube (13). In this case, seal member (23) prevents leakage between valve body (21) and the inner circumferential surface of small diameter part (11 a).

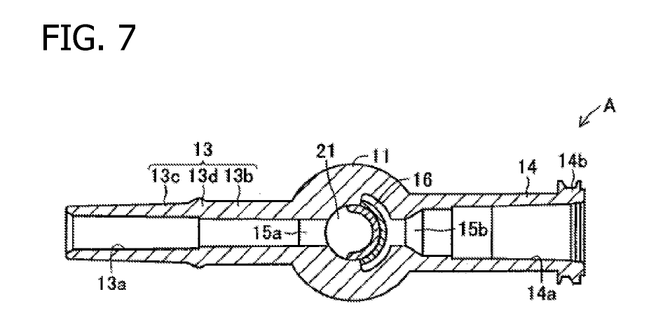

A curved surface (21a) that extends curving downward from flow merging branch tube (12) toward downstream branch tube (13) is formed in the section at the top of valve body (21) facing downstream branch tube (13). The curved surface (21a), as shown in FIGS. 5 and 7, is formed such that when valve body (21) is at the bottom position, the surface bottom end is positioned at the bottom end of channel (13a) in downstream branch tube (13) and the top end is separated from rubber stopper (17). And as shown in FIG. 3, curved surface (21 a) is formed such that when valve body (21) is at the top position, the surface bottom end is positioned somewhat above the top end of channel (13a) in downstream branch tube (13) and the top end presses against rubber stopper (17).

For this reason, as shown in FIG. 3, when valve body (21) is at the top position, slit (12a) in flow merging branch tube (12) and channel (14a) in upstream branch tube (14) are both disconnected from chamber part (11). Although channel (13a) in downstream branch tube (13) is connected to chamber part (11), it is disconnected from slit (12a) in flow merging branch tube (12) and channel (14a) in upstream branch tube (14). Also, as shown in FIG. 5, when valve body (21) is in the down position, channel (13a) in downstream branch tube (13) and channel (14a) in upstream branch tube (14) are both connected to chamber part (11) and connected to each other.

Therefore, in this state, medicine or the like can be sent from upstream branch tube (14) through chamber part (11) to downstream branch tube (13). A mark (24a) that indicates that the channel inside chamber (11) is turned from flow merging branch tube (12) toward downstream branch tube (13) is furnished on the section of the circumferential surface of valve body (21) at window (18) when valve body (21) is in the up position. Specifically, the mark (24a) indicates the state in which upstream branch tube (14) is disconnected from chamber part (11) and flow merging branch tube (12) can be connected to chamber part (11). A mark (24b) that indicates that the channel in chamber (11) is turned from upstream branch tube (14) toward downstream branch tube (13), and is turned from upstream branch tube (14) toward downstream branch tube (13) [sic] is furnished on the section of the circumferential surface of valve body (21) positioned at window (18) when valve body (21) is in the down position.

Grip part (22) is constituted with a disk shaped bottom part (22a) that extends horizontally from the bottom end of valve body (21), and a cylindrical grip part body (22b) that extends upward along the outer circumferential surface of small diameter part (11a) of chamber part (11) from the outer circumferential edge of bottom part (22a). A guide slot (25) as the guided part of the present invention is formed in the section of grip part body (22b) corresponding to guiding projection (19). The guide slot (25) is formed with a width such that guiding projection (19) can move inside it along its length, and is constituted with a vertical guide (25a) that extends vertically, and horizontal guides (25b) and (25c) that extend horizontally parallel from the top end and bottom end, respectively, of vertical guide (25a).

Narrowed parts (26a) and (26b) are respectively formed at the ends of horizontal guides (25b) and (25c). The narrowed parts (26a) and (26b) are constituted with an elastic part such that the width of the narrowest part is somewhat smaller than the diameter of guiding projection (19). With a specific pressure, guiding projection (19) can be passed through this part by rotating operating part (20) so that guiding projection (19) moves from the center part to the end of horizontal guides (25b) and (25c). When guiding projection (19) is positioned at the end of horizontal guides (25b) and (25c), however, guiding projection (19) is securely held in that position by narrow parts (26a) and (26b).

Also, as shown in FIGS. 8-11, an adaptor (27) is detachably attached to flow merging branch tube (12). The adaptor (27) is constituted with a cylindrical female luer part (27a) in which a channel is formed on the inside, a narrow diameter cylindrical insertion part (27b) as the luer member of the present invention that has a channel that connects with the channel in female luer part (27a), and a cover part (27c) that covers the perimeter of insertion part (27b). A male luer part connected to a transfusion tube (not shown) is inserted into female luer part (27a) and medicine or the like is sent through the transfusion tube. Female luer part (27a) and the inside of chamber part (11) are connected by inserting insertion part (27b) into slit (12a) in rubber stopper (17).

Cover part (27c) is constituted with a cylindrical body that extends downward to cover the circumferential surface of insertion part (27b), maintaining a constant spacing after widening horizontally from the bottom end of female luer part (27a). A pair of locking holes (28a) and (28b) that can lock onto projections (12c) and (12d) of flow merging branch tube (12) is formed in the facing sections near the bottom of the circumferential surface of cover part (27c). By locking the locking holes (28a) and (28b) onto projections (12c) and (12d) with insertion part (27b) inserted into slit (12a), insertion part (27b) can remain inserted into slit (12a). Also, in this case, insertion part (27b) and the circumferential surface of slit (12a) are pressed tightly together due to the elasticity of rubber stopper (17).

In this constitution, to supply a specific medicine to a patient (not shown), the back end of a transfusion tube (not shown), connected to an indwelling needle which pierces and remains in the patient, is connected to downstream branch tube (13). A male luer part, furnished at the tip of a transfusion tube that extends from a container or the like storing the medicine to be supplied to the patient, is connected to upstream branch tube (14). Then, with the indwelling needle inserted and remaining in the patient, the medicine is administered to the patient by operating the operating part (20) to send the medicine from the container or the like to the patient. To administer another medicine or the like to the patient in addition to medicine supplied from a container or the like, the other medicine is injected into chamber part (11) from flow merging branch tube (12) through a transfusion tube connected to adaptor (27).

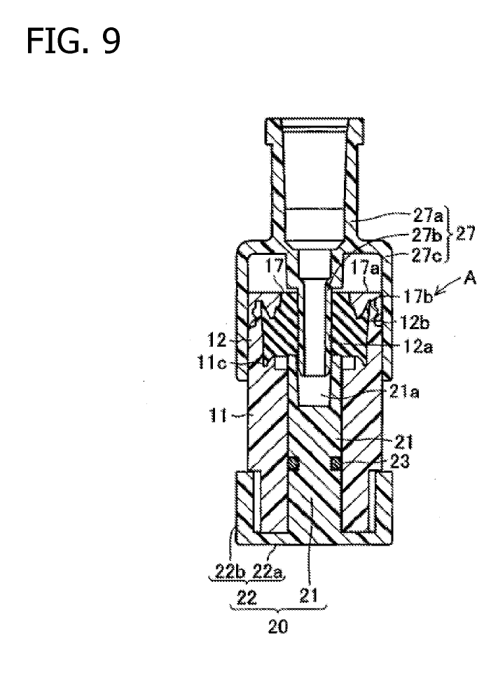

That is, by operating the operating part (20) so that guiding projection (19) is positioned at the end of horizontal guide (25c) with adaptor (27) attached to flow merging branch tube (12), as shown in FIGS. 8 and 9, female luer part (27a) of adaptor (27) and downstream branch tube (13) are connected through chamber part (11). In this case, upstream branch tube (14) is disconnected from the inside of chamber part (11) because the top end of valve body (21) is tight against rubber stopper (17). Mark (24a) that indicates that flow merging branch tube (12) and downstream branch tube (13) are connected appears in window (18) of chamber part (11), as shown in FIG. 1 (b). In addition, projection (19) is secured at the end of horizontal guide (25c) by narrow part (26b).

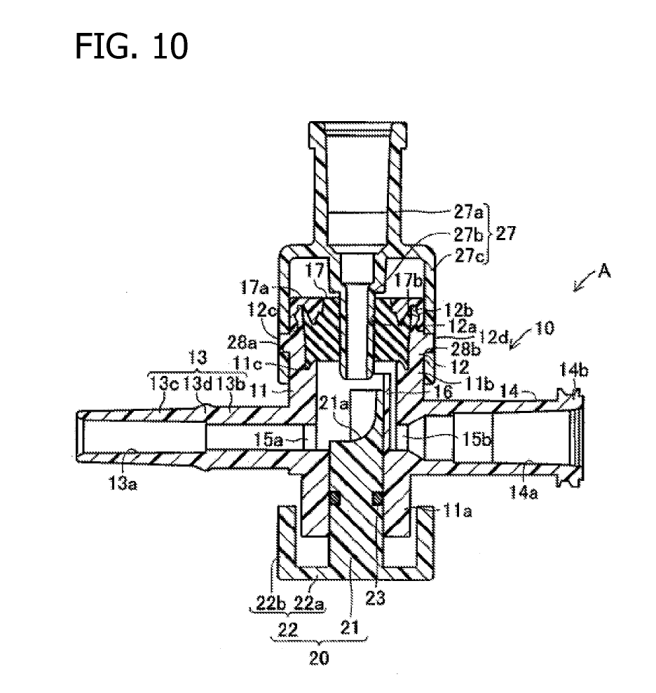

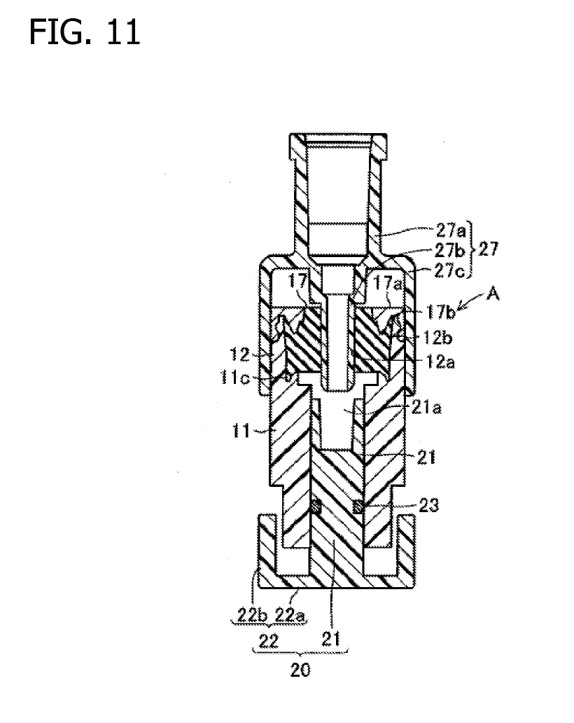

When operating part (20) is operated so that guiding projection (19) is positioned at the end of horizontal guide (25b), as shown in FIGS. 10 and 11, female luer part (27a) of adaptor (27), downstream branch tube (13) and upstream branch tube (14) are all connected through chamber part (11). At this time, the top end of valve body (21) is separated from rubber stopper (17). To indicate that flow merging branch tube (12) and downstream branch tube (13), and upstream branch tube (14) and downstream branch tube (13) are connected, mark (24b) appears in window (18) of chamber part (11), as shown in FIG. 2(b). In addition, guiding projection (19) is secured at the end of horizontal guide (25b) by narrow part (26a).

Here, operation of operating part (20) in this case is accomplished from the state in FIG. 1(b), by turning operating part (20) clockwise, as viewed from the bottom surface, so that guiding projection (19) is positioned at the bottom end of vertical guide (25a), and also moving operating part (20) downward so that guiding projection (19) is positioned at the top end of vertical guide (25a). Then operating part (20) is turned counterclockwise, as viewed from the bottom surface, so that guiding projection (19) is positioned at the end of horizontal guide (25b), yielding the state in FIG. 2(b). The reverse operation is performed to go from the state in FIG. 2(b) to the state in FIG. 1(b).

In this way, with liquid co-infusion device (A) pertaining to this embodiment, an operating part (20) is provided that can move up and down in the axial direction of chamber part (11). By manipulating operating part (20) up and down, downstream branch tube (13) and upstream branch tube (14) can be connected and disconnected by valve body (21). It is also constituted so that an adaptor (27) can be detachably attached to flow merging branch tube (12) formed at the top of chamber part (11), and adaptor (27) can be connected to the inside of chamber part (11) by attaching adaptor (27).

Therefore, one or two types of medicine or the like can be selected and administered as appropriate to the patient. With liquid co-infusion device (A), the operation to switch between connection and disconnection of downstream branch tube (13) and upstream branch tube (14) is accomplished by moving operating part (20) up and down, so it is simple and mishandling is also unlikely to occur. In this case, operating part (20) only advances or retreats relative to chamber part (11), so that operating part (20) does not project from the circumferential surface of chamber part (11) to get in the way. In addition, when liquid co-infusion device (A) is placed on a bed or the like, the axial direction of chamber (11) is horizontal, so that operating part (20) will not contact the patient causing the operating position to be switched.

Also, with liquid co-infusion device (A) pertaining to the present invention, a guide slot (25) is furnished in grip part (22) of operating part (20), and a guiding projection (19) is also furnished in small diameter part (11a) of chamber part (11). Operating part (20) moves relative to chamber part (11) by moving guide slot (25) relative to guiding projection (19), allowing switching between connection and disconnection of downstream branch tube (13) and upstream branch tube (14). For this reason, operating part (20) moves along a precise path and channel switching is reliable.

Operating part (20) also has a grip part (22) that can move along the outer circumferential surface of chamber part (11), so that operation of operating part (20) is easy. In addition, narrow parts (26a) and (26b) are furnished near the two ends of guide slot (25), so that guiding projection (19) is securely locked by narrow parts (26a) and (26b), so that it will not move. Because of this, guiding projection (19) is prevented from moving unintentionally and the channel from being changed. A window (18) is also formed in chamber part (11), and marks (24a) and (24b) are furnished on the section of the circumferential surface of valve body (21) exposed in window (18) to indicate the connected channels when valve body (21) is positioned toward the inside in the axial direction of chamber part (11), and on the section exposed in window (18) when valve body (21) is positioned toward the outside in the axial direction of chamber part (11), respectively. For this reason, the operator will not confuse the channels that are connected or disconnected.

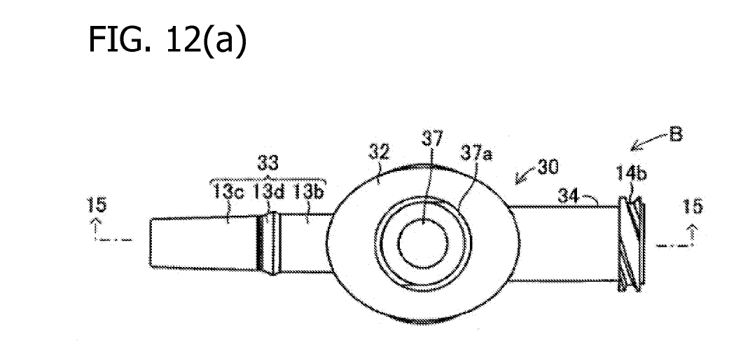

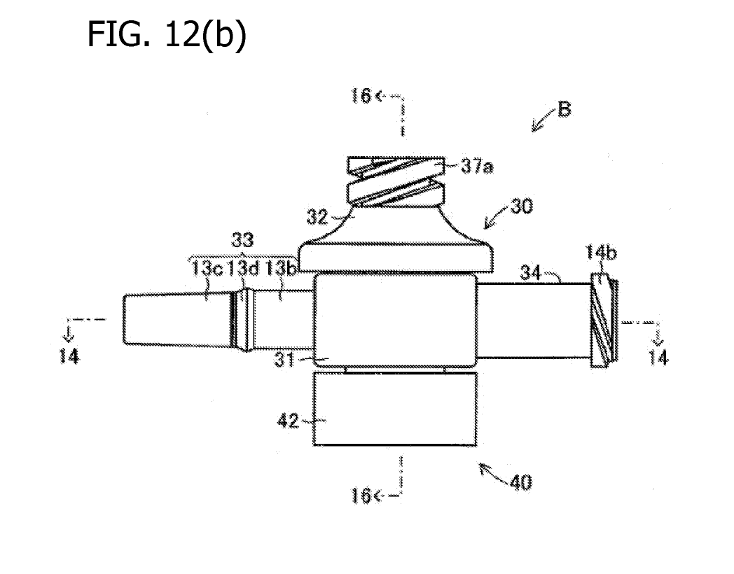

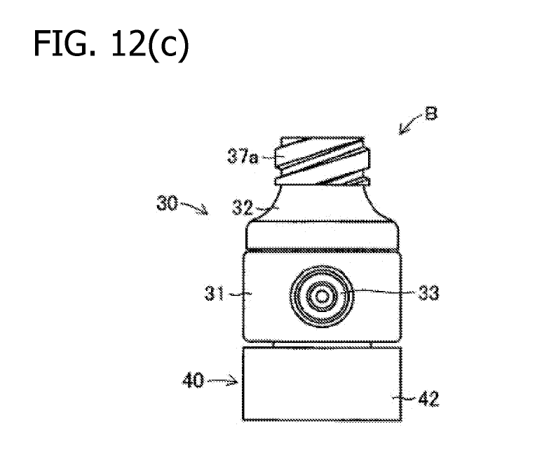

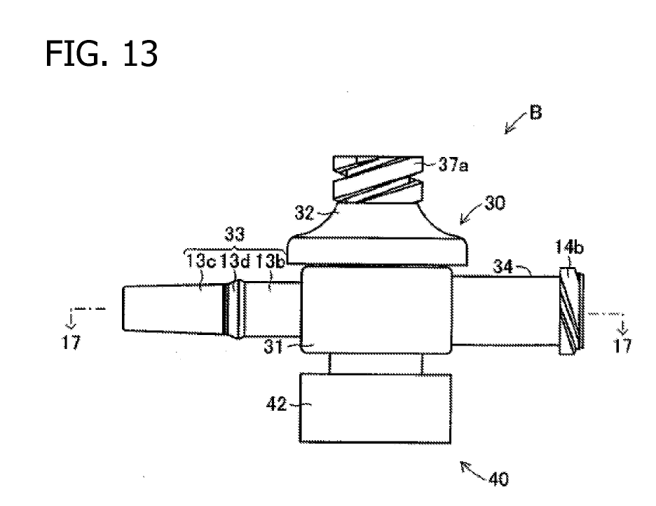

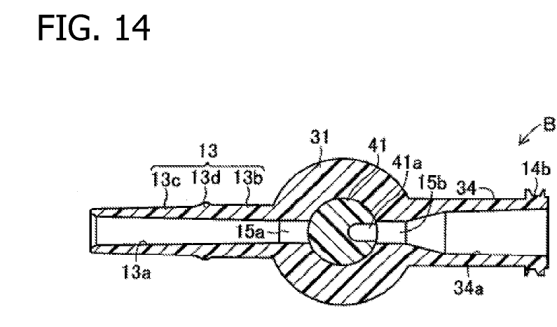

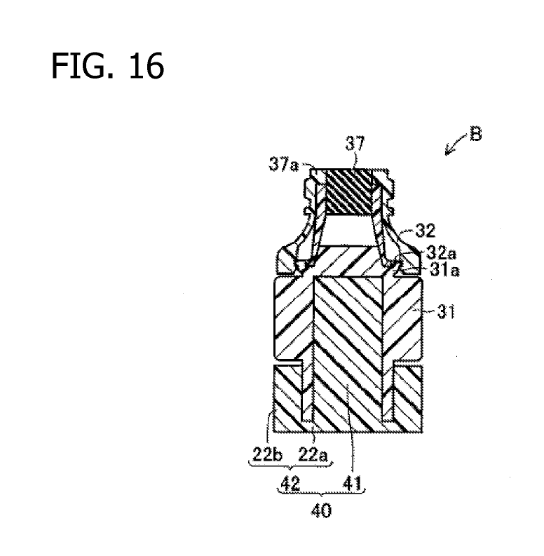

FIGS. 12 and 13 show a liquid co-infusion device (B) pertaining to a second embodiment of the present invention. The liquid co-infusion device (B) is constituted with a liquid co-infusion device body (30) and an operating part (40), and the inside is constituted as shown in FIGS. 14-18. Specifically, flow merging branch tube (32) formed at the top of chamber part (31) is constituted with a member that is separate from chamber part (31) and is constituted with a tapered cylinder body such that the diameter toward chamber part (31) is larger and the diameter becomes smaller going upward. The shape of flow merging branch tube (32), in plan view, is elliptical.

A lockable part (32a) is formed at the bottom end on the inner circumferential surface of merging branch tube (32), and merging branch tube (32) is connected to chamber part (31) by locking lockable part (32a) onto locking part (3 la) formed on the outer circumferential surface at the top end of chamber part (31). A rubber stopper (37) is inserted into the opening at the top end of merging branch tube (32), and rubber stopper (37) is secured from coming loose from the opening in merging branch tube (32) by cover (37a). A channel (37b) is formed in rubber stopper (37) that extends upward from the outer circumferential section of the lower surface, and rubber stopper (37) is locked to the top end of merging branch tube (32) by inserting the top end of merging branch tube (32) into groove (37b).

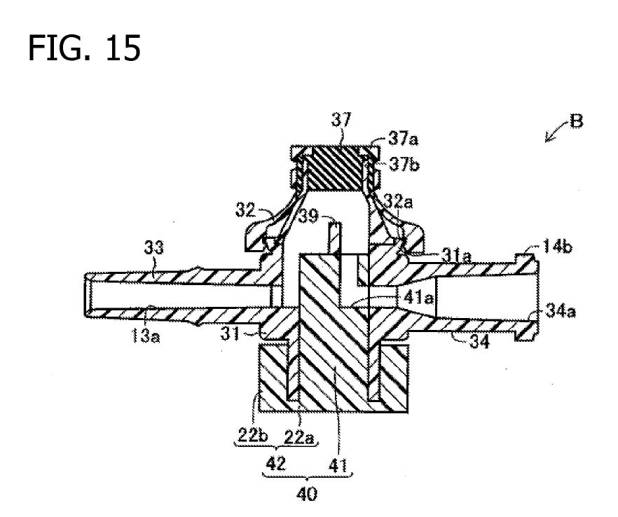

Rubber stopper (37) is secured to the top of flow merging branch tube (32) by tightening the outer circumferential part of the top surface and the top part of the circumferential surface using cover (37a). Here, a slit (not shown) for forming a channel in flow merging branch tube (32) is also furnished in rubber stopper (37). A wall (39) is formed inside chamber part (31) in a section toward the top. Medicine or the like flowing inside chamber part (31) passes through the top of the interior of chamber part (31) when in the state shown in FIG. 15 because of wall (39). Because of this, air or the like can be prevented from remaining in chamber part (31).

Operating part (40) is constituted with a valve body (41) that is approximately a round column, and a grip part (42) that is connected to the bottom end of valve body (41). A channel (41a) in the section of valve body (41) facing upstream branch tube (34) turns at a right angle, after extending from upstream branch tube (34), toward downstream branch tube (33) to extend upward toward flow merging branch tube (32). The bottom end of channel (41a), as shown in FIG. 15, is at a position wherein it connects to channel (34a) in upstream branch tube (34) when valve body (41) is in the up position, and upstream branch tube (34) is connected to the inside of chamber part (31). In this case, the top end surface of valve body (41) touches wall (39).

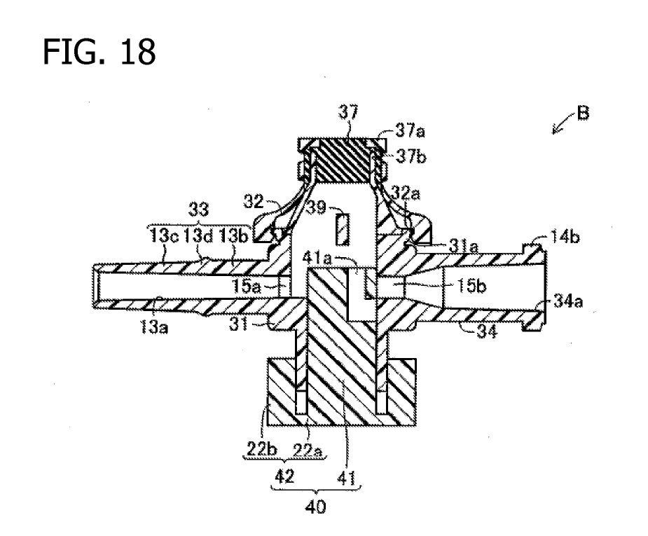

As shown in FIG. 18, the bottom end of channel (41 a) is below channel (34a) of upstream branch channel (34) when valve body (41) is in the down position, and the channel is blocked by the inner circumferential surface of chamber part (31). In this case, the top end surface of valve body (41) is separated from and below wall (39). For this reason, as shown in FIG. 15, when valve body (41) is in the up position, downstream branch tube (33) and upstream branch tube (34) are connected through chamber part (31). Also, as shown in FIG. 18, when valve body (41) is in the down position, downstream branch tube (33) and upstream branch tube (34) are disconnected by valve body (41).

An adaptor that is the same as adaptor (27) in the abovementioned first embodiment can also be detachably attached to flow merging branch tube (32). Here, no window, marks, guide slots or guiding projections are furnished for liquid co-infusion device (B). Operating part (40) in liquid co-infusion device (B) is placed in a specific position using the frictional resistance between it and chamber part (31). The constitution of the other sections of liquid co-infusion device (B) is the same as for said liquid co-infusion device (A). Therefore, the same symbols denote the same sections, and explanations are omitted.

In this constitution, when a specific medicine is administered to a patient, a transfusion tube connected to an indwelling needle is connected to downstream branch tube (33) and a transfusion tube, extending from a container or the like in which a medicine is stored, is connected to upstream branch tube (34). Then, with the indwelling needle inserted and remaining in the patient, operating part (40) is operated and medicine is administered to the patient by sending the medicine from the container or the like toward the patient. When another medicine or the like is administered to the patient in addition to the medicine supplied form the container or the like, the other medicine is injected into chamber (31) from flow merging branch tube (32) through a transfusion tube connected to the adaptor.

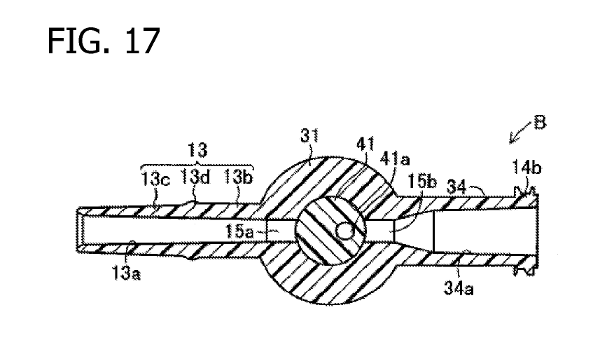

That is, when operating part (40) is in the up position, with the adaptor detached from flow merging branch tube (32), as shown in FIGS. 14-16, channel (41a) in valve body (41) is connected to channel (34a) in upstream branch tube (34), and upstream branch tube (34) is connected to chamber part (31). When operating part (40) is in the down position, as shown in FIGS. 17 and 18, channel (41 a) in valve body (41) is blocked by the inner circumferential surface of chamber part (31), and upstream branch tube (34) and chamber part (31) are disconnected.

In addition, when the adaptor is attached to converting branch tube (32), the adaptor and downstream branch tube (33) can be connected through chamber part (31). In this case, when channel (41a) in valve body (41) and channel (34a) in upstream branch tube (34) are left connected, medicine or the like can be supplied to downstream branch tube (33) from both the adaptor and upstream branch tube (34). When channel (41a) in valve body (41) and channel (34a) in upstream branch tube (34) are left disconnected, liquid medicine or the like can be supplied to downstream branch tube (33) only from the adaptor. Since liquid co-infusion device (B) is constituted as described above, the structure is simple and operation is also simple. Otherwise, the functioning and effects of liquid co-infusion device (B) are the same as in the abovementioned liquid co-infusion device (A).

The liquid co-infusion device pertaining to the present invention is not limited to the abovementioned embodiments, and modifications can be implemented as appropriate. For example, with the abovementioned first embodiment, guide slot (25) is constituted with a vertical guide (25a) that extends vertically and horizontal guides (25b) and (25c) that extend in parallel horizontally, and narrow parts (26a) and (26b) are formed at the ends of horizontal guides (25b) and (25c), respectively, but the guide slot could also be constituted with only a vertical guide (25a). In this case, a narrow part is formed near each end of vertical guide (25a). The guide groove can also be constituted with a slot that is furnished only in the inner circumferential surface of grip part (22) rather than with a long hole through the inner and outer surfaces of grip part (22), so as not to be visible from the outside.

In addition, the guide slot and guiding projections are in facing sections of the chamber part and the operating part, but they can also be furnished in other sections. The guiding part and guided part can also be constituted with other than a guide slot and a guiding projection. In addition, a transparent member can also be furnished for window (18) so that the inside is visible. With the abovementioned application examples, a slit (12a) or the like is furnished in rubber stopper (17) or (37) and insertion part (27b) is inserted into slit (12a), with an adaptor (27) or the like being attached to flow merging branch tube (12) or (32), but the male luer part of a syringe or an injection needle can also be inserted into rubber stopper (17) or (37) in place of adaptor (27). Here, when an injection needle is inserted, it is not necessary to provide slit (12a) in rubber stopper (17) or (37). In addition, the shape, materials, and the like of other sections that constitute the liquid co-infusion device can also be modified and implemented as appropriate.

At least one of the above-described embodiments of the present invention provides a liquid co-infusion device with which any of multiple branch tubes can be connected or disconnected without the operating part projecting and getting in the way.

A feature in the constitution of the liquid co-infusion device pertaining to at least one embodiment of the present invention is that it has: a liquid co-infusion device body composed of a chamber part formed in the shape of a tube, a branch tube formed at one end in the axial direction of the chamber part and that has a chamber that can be connected to or disconnected from the interior of the chamber part, and a pair of branch tubes that extend outward from two sides of the outer circumferential surface of the chamber part and each of which has a channel that connects to or disconnects from the interior of the chamber part; and an operating part that is arranged at the other end in the axial direction of the chamber part to be movable in the axial direction of the chamber part, and which has a valve body that switches between connection or disconnection of one of the pair of branch tubes and the interior of the chamber part by being positioned toward the interior of the chamber part or toward the outside of the chamber part.

The liquid co-infusion device of at least one embodiment of the present invention that is constituted as described above has an operating part with a valve body that can move in the axial direction of the chamber part. By operating the operating part, one of the pair of branch tubes and the interior of the chamber part can be connected or disconnected. The branch tube formed at one end in the axial direction of the chamber part has a channel that can be connected to or disconnected from the interior of the chamber part. Therefore, by operating the operating part, the pair of branch tubes can be connected through the chamber part, and by connecting the channel of the branch tube at one end of the chamber part to the chamber part, the three branch tubes can all be connected through the chamber part.

Because of this, two types of medicine or the like can be administered to the patient. Also, by disconnecting the channel of a branch tube at one side of the chamber part from the chamber part in this state, only a pair of branch tubes can be connected. In addition, by connecting one of the pair of branch tubes and the interior of the chamber part with the channel in the branch tube at the end of the chamber part connected to the chamber part, the branch tube at the end of the chamber part and the other of a pair of branch tubes can be connected. In this case, the pair of branch tubes could also be connected to each other when the valve body is positioned toward the inside in the axial direction of the chamber part, and a pair of branch tubes can also be connected to each other when the valve body is positioned toward the outside in the axial direction of the chamber part.

Using this liquid co-infusion device, switching between connection and disconnection of the pair of branch tubes is accomplished just by moving the operating part in the axial direction of the chamber part. This is simple, and the position of the operating part is also easy to determine visually, so mishandling does not occur easily. Also, the operating part only advances or withdraws in the axial direction relative to the chamber part, so it does not project from the circumferential surface of the chamber part to get in the way. In addition, when this type of liquid co-infusion device is placed on a bed or the like, the axial direction of the chamber part is oriented horizontally, so the position in which the operating part can be switched by contact with the patient also does not occur.

Here, the shape of the chamber part in this case may be that of a round tube, a square tube, a triangular tube, an elliptical tube, a semicircular tube, or any other tube shape that extends in the axial direction. The valve body used may be formed by cutting away one side of a round columnar body such that it can close only one of the pair of branch tubes, or by forming a channel composed of a hole inside a round columnar body.

Another feature in the constitution of a liquid co-infusion device pertaining to at least one embodiment of the present invention is that a guiding part is furnished in the section of the chamber part where the operating part is disposed, a guided part is furnished for the operating part that can move relative to the guiding part when locked to the guiding part, and the operating part moves relative to the chamber part by moving the guided part relative to the guiding part.

Because of this, the operating part moves in a precise path formed by the relative movement with the guiding part and the guided part locked, so that channel switching is reliable. In this case, the guiding part and the guided part can be constituted with, for example, a slot and a locking projection that can move while sliding locked within the slot, and the guiding part and the guided part could be furnished in either the chamber part or the operating part. In addition, the guiding part and the guided part could also be furnished at any section of the chamber part of the operating part. In short, the chamber part and the operating part need only be able to move relatively via the guiding part and the guided part.

Still another feature in the constitution of a liquid co-infusion device pertaining to at least one embodiment of the present invention is that the operating part has a grip part that can move in the axial direction along the outer circumferential surface of the section on said other end, in the axial direction, of the chamber part. Because of this, the operator operating the liquid co-infusion device holds the grip part to move the operating part in the axial direction of the chamber part so that switching is performed using the valve body. Also, in this case, because the grip part can move along the outer circumferential surface of the chamber part, operation is easy.

Still another feature in the constitution of a liquid co-infusion device pertaining to at least one embodiment the present invention is that the guiding part is furnished on the outer circumferential surface in a section at said other end, in the axial direction, of the chamber part, and the guided part is furnished in a section of the grip part corresponding to the guiding part. Because of this, formation of the guiding part and the guided part is easy. The operating part moves along a precise path by movement of the guided part along the guiding part, and channel switching is reliable.

Still another feature in the constitution of a liquid co-infusion device pertaining to at least one embodiment of the present invention is that the guided part is constituted with a slot or a through hole that has a section that extends in the axial direction of the chamber part, and the guiding part is constituted with a projection that locks into the slot or through hole to be able to move in it. Also, in this case, a narrow part for positioning the projection at the two ends of the slot or through hole is furnished near the two ends of the slot or the through hole that constitutes the guiding part. Because of this, the projection locks in the narrow part and is restrained, so that the channel is prevented from being changed by unintentionally moving the projection.

Still another feature in the constitution of a liquid co-infusion device pertaining to at least one embodiment of the present invention is that a window to expose the circumferential surface of the valve body is formed in a specific section of the chamber part, and marks that indicate the channel that will be connected are furnished on the circumferential surface of the valve body at the section exposed to the window when the valve body is positioned toward the inside in the axial direction of the chamber part and at the section exposed to the window when the valve body is positioned toward the outside in the axial direction of the chamber part. Because of this, the channel is indicated in the window, and the channels that are to be connected or disconnected are not operated incorrectly.

Still another feature in the constitution of a liquid co-infusion device pertaining to at least one embodiment of the present invention is that a rubber stopper is attached to the branch tube formed at one end in the axial direction of the chamber part, and by passing a tubular insertion member into the rubber stopper, the interior of the chamber part and a liquid container connected to the tubular insertion member are connected through the branch tube formed at one end in the axial direction of the chamber part. Because of this, the channel in the branch tube formed at one end of the chamber part can be connected to or disconnected from the interior of the chamber part in a simple fashion. The tubular insertion member in this case is the inserted part (dull needle) of an adaptor, the male part of a luer syringe, or the like. When the tip of the tubular insertion member is not sharp, a slit for passing the tubular insertion member through can also be provided in the rubber stopper.

Having described the embodiments of the invention in detail, it will be apparent that modifications and variations are possible without departing from the scope of the invention defined in the appended claims.

When introducing elements of the present invention or the illustrated embodiments thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A liquid co-infusion device having a body comprising:
a chamber, having a first connection port formed at one end in an axial direction of said chamber, and second and third connection ports extending sidewards from said chamber,
and an operating part, arranged at a second end in the axial direction of said chamber and movable in the axial direction of said chamber, wherein movement of the operating part in said axial direction switches between connection and disconnection of one of said connection ports to an interior of said chamber.

2. The liquid co-infusion device according to claim 1, wherein said operating part includes an outer sidewall arranged to be slidably movable over a outer surface portion of said chamber and wherein said outer sidewall and said outer surface portion include mutually co-operating guide parts arranged so as to guide the movement of said operating part.

3. The liquid co-infusion device according to claim 2, wherein the mutually co-operating guide parts comprise a guide slot and a projection arranged to move in said guide slot.

4. The liquid co-infusion device according to claim 3 wherein said guide slot is arranged to include an axially extending part and at least one part extending laterally to said axially extending part so as to selectively allow axial movement of said operating part and to prevent axial movement of said operating part respectively.

5. The liquid co-infusion device according to claim 1 wherein a window is provided in a wall of said chamber and wherein said operating part includes markings thereon such that an operating state of said co-infusion device is displayed in said window.

6. The liquid co-infusion device according to claim 1 wherein said operating part includes a through channel therein, wherein movement of said operating part in said axial direction displaces said through channel between a fluid flow position relative to one of said connection ports and a fluid sealing position relative to said one connection port.

7. The liquid co-infusion device according to claim 1, wherein said operating part includes a groove at an end thereof disposed within said chamber and wherein said groove is bounded by a sidewall such that when said operating part is moved in said axial direction into said chamber, said sidewall provides a fluid flow seal to cause said disconnection.

8. The liquid co-infusion device according to claim 7, wherein one of said connection ports incorporates an elastic sealing element and said operating part sidewall is moveable to provide a fluid flow blocking seal between said sidewall and said sealing element.

9. The liquid co-infusion device according to claim 8, wherein said groove is shaped to accommodate an end of a connector inserted through said elastic sealing element when said operating part is positioned to as to cause said disconnection.
